(19) United States
(12) Patent Application Publication
(10) Pub. No.: US 2009/0280099 A1
(43) Pub. Date: Nov. 12, 2009

(54) NASOPHARYNGEAL INOCULATE OF PROBIOTICS AND PREBIOTICS FOR TREATMENT OF RESPIRATORY INFECTIONS

(76) Inventors: Stephen E. Bachman, Armarillo, TX (US); Jeremy J. Mathers, Tinley Park, IL (US)

Correspondence Address: KING PHARMACEUTICALS, INC., 400 CROSSING BOULEVARD, BRIDGEWATER, NJ, 08807, US

(21) Appl. No.: 12/435,763

(22) Filed: May 5, 2009

Related U.S. Application Data
(60) Provisional application No. 61/051,291, filed on May 7, 2008.

(51) Int. Cl. A61K 35/74; A61P 31/00; A61P 11/00

(52) U.S. Cl. 424/93.45; 424/93.4; 424/93.46

(57) ABSTRACT

The present invention is directed to compositions comprising one or more species of probiotic bacteria, optionally in combination with one or more prebiotics, to methods of preventing and treating respiratory infections in animals by employing such compositions, and to methods of preparing the same.

This application claims the benefit of U.S. Provisional Application No. 61/051,291 filed May 7, 2008, incorporated herein by reference in its entirety.

### BACKGROUND OF THE INVENTION

Probiotic bacteria are generally known to be clinically safe (i.e. nonpathogenic). Recent experimental evidence suggests that the ingestion of substantial numbers of beneficial bacteria can offer positive effects to animals.

An infection of the lungs that involves the small air sacs or alveoli and the tissues around them is known as pneumonia. Over two million people are known to develop this infection each year with between 40,000 to 70,000 pneumonia-related deaths per year. Recent studies have shown there are an increasing number of bacteria developing which cause pneumonia that are antibiotic resistant making this the sixth most common cause of death over all. The term pneumonia covers a variety of illnesses with each being caused by a different microscopic organism. In most cases the organisms are inhaled through the lungs but they can also be carried to the lungs in the blood stream or migrate from other infections close to the lungs.

Bovine respiratory disease complex (BRDC) is a complex involving environmental, viral, and bacterial factors. Pneumonia typically starts to appear soon after a significant environmental, physiological, or psychological stress and/or exposure to new disease organisms. Production examples include weaning, commingling at points-of-sale followed by shipping, increasing dietary energy-density in the feedlot, parturition and lactation in dairy cattle, and heat or cold stress in pasture cattle.

BRDC is the biggest health challenge facing today's feedlot—and it is a major cause of economic losses for dairymen. BRDC is estimated to cost the U.S. feedlot more than $500 million each year. Incidence of the disease is approximately 20% of the 25 million cattle placed in U.S. feedlots annually. Mortality in the sick cattle ranges from 10% to 15%, depending on the time of year and other variables.

Several different pathogens play a role in BRDC. Mannheimia (formerly known as Pateurella) haemolytica, biotype A, serotype 1 (A1) is the primary, but not the sole, bacterial pathogen in BRDC in the United States. Mannheimia haemolytica occurs in the nasopharnyx of healthy cattle, and is pathogenic only in ruminants, presumably because of the ability of a heat-labile leukotoxin that is active only against ruminant leukocytes. Mannheimia haemolytica A2 appears to be the primary serotype colonizing the nasopharnyx in cattle on the farm, whereas Mannheimia haemolytica A1 proliferates during shipping and becomes the primary isolate in feedlots. Infection with bovine respiratory viruses enhances nasopharnyx colonization by Mannheimia haemolytica A1, even in the face of active immunity. Colonization of the nasopharnyx allows inspiration of Mannheimia sp. organisms, which are cleared from the lungs if a competent immune system is present. High-stress environments, viral immunosuppression, or inadequate nutrition in the form of energy, protein, and trace elements may allow inspired Mannheimia sp. to colonize the lungs.

Pateurella multocide is secondary in importance to Mannheimia haemolytica in bovine pneumonia except in veal and dairy calves, where prevalence of P. multocida infection may be higher than P. haemolytica. Haemophilus somnus is a major cause of respiratory disease in fall-weaned calves shipped to feedlots in Canada. Mycoplasma organisms play a secondary, but perhaps important, role in bovine respiratory disease.

Viral isolates from bovine respiratory disease cases include 1BRV, bovine viral diarrhea virus (BVDV), bovine respiratory syncytial virus (BRSV), parainfluenza type 3 virus (PI3V), malignant catarrhal fever virus, reovirus, calicivirus, and bovine adenovirus, parvovirus, herpesvirus type 4, rhinovirus, enterovirus, and respiratory coronavirus. Of these IBRV, BVDV, and BRSV are of major importance in field cases of bovine respiratory disease in the United States.

Porcine respiratory disease complex (PRDC) is a major threat to swine operations, despite high-health strategies being adopted today. PRDC is caused by a combination of recognized respiratory pathogens. The primary pathogens include Mycoplasma hyopneumoniae, porcine reproductive and respiratory syndrome (PRRS), swine influenza virus (SIV) and circovirus. Preventing Mycoplasma infection is key to decreasing the impact of PRDC. Prevention and control programs that focus on Mycoplasma have been the most successful in controlling PRDC and, subsequently, PRRS. Pseudorabies virus can have respiratory effects on older pigs—primarily finishing pigs and adult animals. Morbidity is usually high (near 100%), although mortality is low (1-2%). If other respiratory pathogens are present (such as Actinobacillus pleuropneumoniae or swine influenza virus), they will make the respiratory outbreak more damaging to the herd.

Estimates show PRDC affects 10 million pigs a year in the United States, with an economic impact in excess of $40 million annually. Mixed infections from the disease result in a 16-30% reduction in rates of gain and 14-20% reduction in feed conversion.

An estimated 93% of herds worldwide are infected with mycoplasmal pneumonia, making it one of the most prevalent and costly swine diseases.

Equine respiratory disease complex (ERDC) is very common among horses. The stress of hard exercise or transportation, plus exposure to new surroundings and animals, makes the horse vulnerable to viral infection and secondary bacterial invasion by Streptococcus zooepidemicus. Streptococcus species are probably the most common organisms isolated from foals with pneumonia or pulmonary abscesses. In horses, pneumonia can be caused by viruses (equine viral arteritis, equine viral rhinopneumonitis) or bacteria. Often, viral pneumonia weakens a horse, allowing secondary invasion by bacteria. Seventy percent of the horses transported 320 miles developed a respiratory infection within 10 days of delivery.

Feline respiratory disease complex includes those illnesses typified by rhinitis, conjunctivitis, lacrimation, salivation, and oral ulcerations. The principal diseases, Feline Viral Rhinotracheitis (FVR) and Feline Calicivirus (FCV) infections, affect exotic as well as domestic species. Feline Pneurnonitis (Chlamydia psittaci) and Mycoplasmal infections, appear to be of lesser importance. Feline infectious peritonitis and pleuritis typically causes a more generalized condition but may cause signs of mild upper respiratory tract infection. I FVR and calliciviruses are host-specific and pose no known human risk. Human conjunctivitis caused by the feline chlamydial agent has been reported. Probably 40-45% of feline upper respiratory infections are caused by FVRs, which is a herpesvirus; incidence of FCV is similar. Dual infections with these viruses are common. Other organisms such as Chlamydia psittaci, Mycoplasma spp, and reoviruses are believed to account for most of the remaining infections.

Dogs are susceptible to respiratory diseases also, with one such malady sometimes known as “Kennel Cough.” Kennel Cough can be caused by a number of viruses as well as bacteria. Frequently the disease is in fact caused by a combination of these two types of organisms. Primary among the viruses are Canine adenovirus type 1 and 2 as well as Canine parainfluenza virus. Probably the single most important organism in causing kennel cough is a bacterium called Bordatella bronchiseptica.

There remains a need for alternative methods and compositions to treat and/or ameliorate the effects of such respiratory infections.

### BRIEF SUMMARY OF THE INVENTION

The present invention is directed to a method of treating a respiratory infection in an animal which includes administering an effective amount of a probiotic into the nasal passages of the animal.

An additional embodiment of the invention is directed to a method of treating a respiratory infection in an animal which includes administering an effective amount of a prebiotic into the nasal passages of the animal.

A further embodiment of the invention is directed to a method of treating a respiratory infection in an animal which includes administering an effective amount of a prebiotic and a probiotic into the nasal passages of the animal.

Another embodiment of the invention is directed to a composition for preventing or treating a respiratory infection in an animal which includes a probiotic and sodium carboxymethylcellulose.

The invention is also directed to a composition for preventing or treating a respiratory infection in an animal which includes a prebiotic and sodium carboxymethylcellulose (CMC).

### BRIEF DESCRIPTION OF THE DRAWINGS

FIG. 1 shows the total count of ALCARE® spore-averages for treatment and control groups. As seen on the logarithmic scale, the treated group demonstrated very high counts of ALCARE®, when swabbed at post-dose on the same day following nasal injections (error bars indicate the Standard Deviation for 10-cattle groups).

FIG. 2 shows the percentage of total counts having the ALCARE® phenotype for the ALCARE®-exposed group of cattle during the experiment. These data show that while ALCARE® dominated the nasal passage at 99.9% on the same day following injection; the relative percentages fell to 26% at day 1, then further to 14% and 12% at days 3 and 5, respectively (error bars indicate the Standard Deviation for 10-cattle groups).

### DETAILED DESCRIPTION OF THE INVENTION

By way of example, and not of limitation to any particular mechanism, the prophylactic and/or therapeutic effect of probiotic bacteria of the present invention results, in part, from a competitive inhibition of the growth of pathogens due to: (i) their superior colonization abilities; (ii) parasitism of undesirable microorganisms; (iii) the production of extracellular products possessing anti-microbial activity; and/or (iv) various combinations thereof.

As utilized herein, the term “probiotic” refers to microorganisms that form at least a part of the transient or endogenous flora monoculture, and/or a mixed culture of living or dead microorganisms, spores, fractions thereof, or metabolic products thereof, that exhibit a beneficial prophylactic and/or therapeutic effect on the host organism.

The term “effective amount” as used herein refers to the amount of one or more species of probiotic bacteria alone, or in combination with prebiotics, effective to inhibit pathogenic bacterial and mycotic growth in the nasopharnyx.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The compositions and methods of the present invention include bacteria which include non-pathogenic members of the Aspergillus, Bacillus, Bacteroides, Bifidobacterium, Lactobacillus, Leuconostoc, Pediococcus, Propionibacterium, Saccharomyces, and Enterococcus genus which produce bacteriocins or other compounds which inhibit the growth of pathogenic organisms, or occupy sites or receptors within the animal normally colonized by pathogens.

Exemplary Aspergillus bacteria include, but are not limited to, Aspergillus niger and Aspergillus oryzae and genetic variants thereof. Exemplary non-pathogenic Bacillus species include, but are not limited to: Bacillus coagulans, Bacillus lentus, Bacillus licheniformis, Bacillus pumilus, Bacillus subtilis, Bacillus coagulans hammer, and Bacillus brevis subspecies coagulans and genetic variants thereof. Preferred Bacillus species include, but are not limited to the lactic acid-producing Bacillus coagulans and Bacillus laevolacticus.

The compositions and methods of the present invention also include one or more Lactobacillus species, including but are not limited to, Lactobacillus acidophilus, Lactobacillus brevis, Lactobacillus buchneri, Lactobacillus farciminis, Lactobacillus casei, Lactobacillus cellobiosus, Lactobacillus curvatus, Lactobacillus fermentum, Lactobacillus helveticus, Lactobacillus lactis, Lactobacillus plantarum, Lactobacillus DDS-1, Lactobacillus GG, Lactobacillus rhamnosus, Lactobacillus plantarum, Lactobacillus reuteri, Lactobacillus gasserii, Lactobacillus jensenii, Lactobacillus deibruekii, Lactobacillus bulgaricus, Lactobacillus salivarius, Lactobacillus farciminis, and Lactobacillus sporogenes (also designated as Bacillus coagulans) and genetic variants thereof.

The compositions and methods of the present invention additionally include Sporolactobacillus selected from the group comprising all Sporolactobacillus species and genetic variants thereof. A preferred Sporolactobacillus is Sporolactobacillus P44.

The compositions and methods of the present invention further include one or more Bifidiobacterium species, including but are not limited to Bifidiobacterium adolescentis, Bifidiobacterium animalis, Bifidiobacterium bifidum, Bifidiobacterium bifidus, Bifidiobacterium breve, Bifidiobacterium infantis, Bifidiobacterium infantus, Bifidiobacterium longum, Bifidiobacterium thermophilum, and genetic variants thereof.

The compositions and methods of the present invention also include one or more Pediococcus species including, but not limited to, Pediococcus acidilacticii, Pediococcus cerevisiae, and Pediococcus pentosaceus and genetic variants thereof.

The compositions and methods of the present invention may additionally include Propionbacterium acidipropionici, Propionbacterium freudenreichii, Propionbacterium shermanii, and genetic variants thereof.

Saccharomyces cerevisiae may also be included in the compositions and methods of the present invention.

The compositions and methods of the present invention also may include one or more Enterococcus species including, but not limited to, Enterococcus cremoris, Enterococcus diacetylactis, Enterococcus faccium, Enterococcus intermedius, Enterococcus lactis, Enterococcus thermophilus, and genetic variants thereof.

The compositions and methods of the present invention may further include one or more Mannheimia, Pateurella, and Haemonphilus species including, but not limited to, Mannheimia haemolytica, Pateurella multocida, and Haemophilus somnus.

Various other non-lactic acid-producing Bacillus species may be utilized in the present invention so long as they produce compounds or occupy sites which possess the ability to inhibit pathogenic bacterial or mycotic growth. Examples of such suitable non-lactic acid-producing Bacillus include, but are not limited to: Bacillus subtilis, Bacillus uniflagellatus, Bacillus lateropsorus, Bacillus laterosporus BOD, Bacillus megaterium, Bacillus polymyxa, Bacillus sterothermophilus and genetic variants thereof.

Although exemplary of the present invention, Bacillus coagulans or Bacillus licheniformis are only utilized herein as a model for various other acid-producing (e.g., lactic acid) species of probiotic bacteria which may be useful in the practice of the present invention, and therefore is not to be considered as limiting. Furthermore, it is also intended that any of the lactic acid-producing species of probiotic or nutritional bacteria can be used in the compositions, therapeutic systems and methods of the present invention.

The Bacillus species, particularly those species having the ability to form spores (e.g., Bacillus coagulans or Bacillus licheniformis), are a preferred embodiment of the present invention. The ability to sporulate makes these bacterial species relatively resistant to heat and other conditions, provides for a long shelf-life in product formulations, and can efficiently colonize the tissues within the nasopharnyx. Moreover, additional useful properties of many Bacillus species include being non-pathogenic, aerobic, facultative and heterotrophic, thus rendering these bacterial species safe and able to readily colonize the nasopharnyx.

The sporeformers of the Bacillus genus, and in particular Bacillus licheniformis, have been successfully employed for use as probiotics in both people and animals. In certain embodiments, Applicants' composition and method include Bacillus licheniformis, ATCC 10716, and a bacterium derived therefrom and sold in commerce under the trade name ALCARE®, manufactured by Alpharma Inc of Bridgewater, N.J. Both Bacillus licheniformis, and the ALCARE® bacterium, are able to survive and colonize the nasopharnyx in a highly efficacious manner.

The ALCARE® bacterium was derived from a strain line of B. licheniformis, used in the manufacture of a widely used animal health product used worldwide. The ALCARE® bacterium was modified by conventional breeding methods to enhance the spore-forming trait when grown in stainless steel vessels according to GMP guidelines. The ALCARE® strain produces and secretes native amylases and proteases (which break down starches and proteins, respectively), and other enzymes during the course of growth and sporulation in the production cycle, prior to spray drying and blending down to the final (concentrated spore) product. The ALCARE® bacterium is deposited with the UK National Collection of Type Cultures, accession number NCTC 13123. It is not a product of recombinant DNA, and its identification to the species level is based on morphological and biochemical characteristics that meet the criteria defined for Bacillus licheniformis.

An embodiment of the present invention includes Bacillus coagulans, ATCC No.31284 (and new variants or mutants thereof), as a probiotic. This Bacillus species is able to survive and colonize the nasopharnyx in a highly efficacious manner. Additionally, probiotic Bacillus coagulans is non-pathogenic and is generally regarded as safe (i.e., GRAS classification) by the U.S. Federal Drug Administration (FDA) and the U.S. Department of Agriculture (USDA), and by those individuals skilled within the art. Because Bacillus coagulans possesses the ability to produce heat-resistant spores, it is particularly useful for making pharmaceutical compositions which require heat and pressure in their manufacture. Accordingly, formulations that include the utilization viable Bacillus coagulans spores in a pharmaceutically-acceptable carrier are particularly preferred for making and using compositions disclosed in the present invention.

The growth of various Bacillus species to form cell cultures, cell pastes, and spore preparations is generally well-known within the art. The culture and preparative methods for Bacillus coagulans may be readily utilized and/or modified for growth and preparation of the other (lactic) acid-producing bacteria disclosed in the present invention.

The compositions and methods may further include one or more “prebiotics” in combination with one or more probiotics. In certain embodiments, these one or more prebiotics include, for example and without limitation, carbohydrates and more specifically, oligosaccharides. Such oligosachharides may be produced from glucose, galactose, xylose, maltose, sucrose, lactose, starch, xylan, hemicellulose, inulin, or a mixture thereof. Purified commercially available products such as fructo-oligosaccharides contain greater than about 95% solids in the form of oligosaccharides.

It is known that inulin and fructo-oligosaccharides (FOS) are found in approximately 36,000 plants at various levels. Besides inulin and FOS, a number of other compounds have potential as prebiotics such as soybean oligosaccharides (raffinose, stachyose), lactulose, isomalto-oligosaccharides, lactosucrose, gluco-oligosaccharides and palatinose. Others may include tagatose, lactitol, pyrodextrins, galacto-oligosaccharides, and xylo-oligosaccharides.

The composition of the present invention contains a suspension of spores as a probiotic in a suitable carrier. Suitable carriers include, but are not limited to, glycerin, sodium CMC, methylcellulose, natural hydrocolloids, alginic acid, carageenan, locust bean gum, guar gum, gelatin or clays. A preferred carrier is sodium CMC.

Current uses of probiotics and prebiotics are primarily centered on oral consumption for prophylaxis and treatment of the gastrointestinal tract. Applicants have found, however, that nasal administration of one or more probiotics, optionally in combination with one or more prebiotics, can effectively treat diseases of the respiratory tract, such as pneumonia, in animals.

An embodiment of the present invention includes a method of inoculating the nasopharyngeal region of an animal with probiotics and/or prebiotics in a manner to alter the microflora and create an environment that beneficially affects the host. The “nasopharyngeal region” means the nasal or oral cavities or the surrounding regions, including associated ligual, palatine, pharyngeal and esophageal tonsils and lymphoid tissues. In various embodiments of the present invention, the inoculation may comprise a single application. Such a single administration is useful for prophylactic purposes. In other embodiments, the inoculation may comprise multiple applications given over a time period of minutes, days, weeks, or months. Such multiple administrations are useful to treat respiratory diseases in animals, particularly if given early in the development of the disease. In certain embodiments, the inoculate is administered as one or more solids, in one or more liquid carriers, as a gel, and/or as a paste.

The nasopharyngeal administration of the composition of the present invention may be useful in treating respiratory diseases in an animal suffering from a variety of respiratory conditions and may be used in combination with traditional drug therapy. The present invention may be useful in mammals, including, but not limited to humans, cattle, pigs, sheep, cats, and dogs.

### EXAMPLE 1

Sodium CMC, Sigma C-488 (medium viscosity), was weighed and added to buffered deionized water (citrate-phosphate buffer, 50 mM) to prepare solutions of 0.1%, 0.25%, 0.5%, and 1% w/v of sodium CMC carrier solutions. The CMC carrier mixtures were continuously stirred for 3 hours to dissolve the dry sodium CMC into the water. The pH of the buffered water was around 5.5, and the final pH was 7.0 for the sodium CMC carrier solutions. Pre-measured volumes of 50 mL of the sodium CMC carrier solutions were used to suspend ALCARE® (manufactured by Alpharma Inc. of Bridgewater, N.J.) Bacillus licheniformis spore concentrate (minimum of 1×10¹⁰ colony forming units (cfu) per g of ALCARE® concentrate) using a vortex for approximately 2 minutes to suspend the spores. The suspension containing 0.25 g of ALCARE® spore concentrate in 50 mL of a 0.25% w/v sodium CMC carrier solution (0.5×10⁸ cfu/mL) offered the best balance of spore dispersion, minimal settled solids, and near-physiological carrier concentration. The carrier-spore suspension remained essentially suspended after one hour of standing at room temperature, in comparison to a deionized water control which showed visible settling after 15 minutes standing at room temperature. An effective amount of the carrier-spore suspension may then be administered to an animal by nasopharyngeal administration, e.g., administering from 1.0×10⁸ to 1.8×10⁹ cfu of spores per animal, per day.

Alternatively, a reagent kit comprising: 1) two 1 g ALCARE® spore concentrate vials (3×10¹⁰ cfu/g of ALCARE® concentrate); and 2) four 200 mL sodium CMC carrier suspension blanks in pre-sterilized, wide-mouth jars; may be used. The entire contents of each spore concentrate vial are poured into one carrier solution jar, the jar is recapped and shaken intermittently for a minimum of 5 minutes to provide the carrier-spore suspension (1.5×10⁸ cfu/mL).

### EXAMPLE 2

The experiment of Example 2 consisted of taking swab samples from 10 healthy cattle in each of the following treatment groups: nasal carrier only, carrier+ALCARE®, and untreated control. Swab samples were taken at T0 (pre-treatment), T1 (post-treatment), and days 1, 3, and 5 post nasal-injection. The cattle in the treatment group received, with both nostrils injected, 0.6×10⁹ cfu of ALCARE® concentrated spores suspended in a 0.25% w/v sodium CMC carrier per animal. The study protocol is summarized in Table 1.

TABLE 1

| | |
|---|---|
| Schedule of Events | Day 1 Swab then dose and swab post dose, with additional |
| | swabs on day 3 and day 5. Observe animals for 28 days for |
| | adverse reactions or negative health response |
| Treatment Groups | 1) Control; 2) Probiotic carrier only; and 3) Carrier and |
| | probiotic |
| Experimental Design | Random |
| Randomization Procedures | Random number generator to select animals in each |
| | treatment |
| Blinding of Study | Daily observations for health effects to be collected by person |
| | blind to treatment group assignment. |
| Test Animals | Age: Yearlings |
| | Sex: Steers |
| | Breed/Class: Mixed beef breeds |
| | Physiological State: Normal and healthy |
| | Number: 30 |
| | Identification Method: Individual ear tag |
| Study Facilities | Containment Equipment: Single pen per treatment group |
| | Feeding Equipment: 2 times a day adlib via feed bunk |
| | Watering Equipment: Constant flow |
| | Space Allocation Per Animal: 300 sq feet |
| Acclimation of Test Animals | Duration: 28 days |
| | Medication/Vaccination during Acclimation Period: None |
| | Baseline Data Collected Prior to Initiating Injections: Body |
| | weight, observations of health and suitability for the study |
| Treatments | Dosing: 2 mL dose (1.5 × 10⁸ cfu/mL) per nostril |
| | Route of Administration: Nasal passage/nares |
| | Investigational withdrawal period: 0 days |
| Cattle measurements | Body weights Day 1 and Day 28 |
| | Nasal swabs (ventral nasal meatus) on Day 1 (pre and post |
| | dose), Day 3 and Day 5 |
| | Daily observation of general health and for adverse reactions |
| | to treatment with test article for total of 28 days |

Swabs were processed according to the following procedure: 1 mL of sterile peptone saline (0.1 g of soy peptone, 0.85 g of NaCl per liter deionized water) added to each swab carrier, swabs were vortexed thoroughly to release the swab contents to the buffer. Initial platings were done on nutrient agar (Criterion, Hardy Diagnostics, California). Swabs were hydrated, then heat shocked (80° C., 10 minutes). This step was necessary due to the relatively high number of bacterial types present in initial tests, precluding detection of the probiotic spore types. Application of the heat step allowed only spore forming bacteria to survive and grow. The presence of the ALCARE® probiotics spores could therefore be detected and counted at relatively low levels amid a background of naturally occurring spore forming bacteria. Counting criterion were based on colony phenotype following growth on nutrient agar overnight at 38° C. (light beige, slightly rough edged, small to medium size, even color and uniformly round appearance). Counts run on swabs collected immediately following injection, required additional dilution (10-200 times) due to very high levels present. The plating volume was 50 μL. Counts were reported as direct counts. Absolute counts (i.e., in units ALCARE® spores/mL nasal fluid) were further calculated on Excel®, based on an overall 200× factor for dilution, swab volume and plating factors). Absolute counts may be relevant for estimating the numbers of pathogens and competitive exclusion strains that might be present in the bovine respiratory tract.

Total counts, and the percentage of ALCARE® spores/total counts in treated sets were averaged for each treatment and control group. Standard deviations were calculated for each set of 10 to estimate the variability for each group. All calculations were done on an Excel® XP spreadsheet.

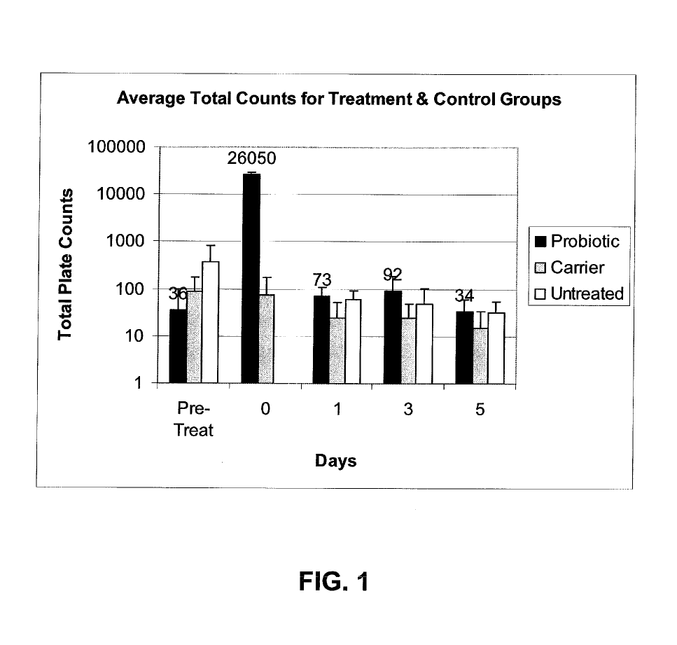

FIG. 1 shows the total count averages for treatment and control groups. As seen on the logarithmic scale, the treated group demonstrated very high counts of ALCARE®, when swabbed at T1 on the same day following nasal injections. These levels translate to ca. 5 million ALCARE® spores per mL nasal fluid in absolute counts. The numbers declined rapidly over 2 log ranges after day 1, consistent with the expected clearance of foreign bodies in the respiratory tract, within 24 hours in healthy animals (The Respiratory System, Thoracid Cavity and Pleura, Thomson's Special Veterinary Pathology, 3^{rd} Ed., Carlton, McGavin, Zachary, Eds. Mosby. 2000, p. 3). The swab tests on controls, showed that there was a low-level presence of spore forming bacteria in all groups, however. This is to be expected in animals eating and breathing near hays, feeds, manures, etc. being constantly exposed to environmental sources.

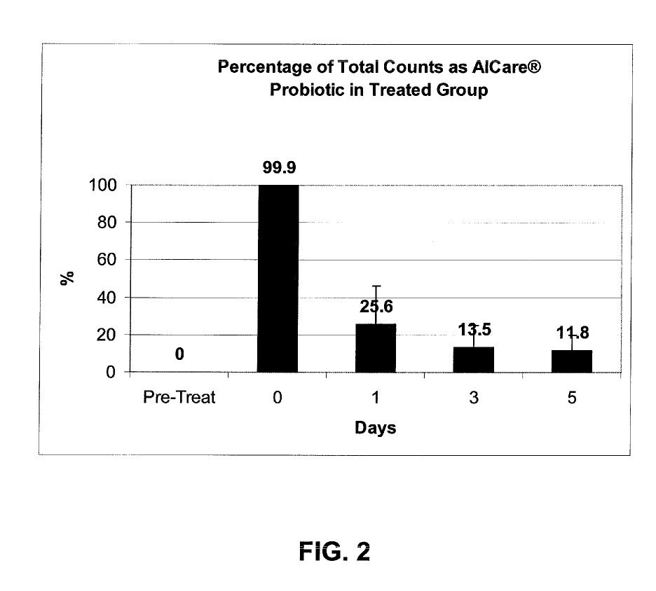

FIG. 2 shows the percentage of total counts having the ALCARE® phenotype for the ALCARE® exposed group of cattle during the experiment. These data show that while ALCARE® dominated the nasal passage at 99.9% on the same day following injection; the relative percentages fell to 26% at day 1, then further to 14% and 12% at days 3 and 5, respectively. In absolute count terms, the amount of ALCARE® declined from ca. 5 million to ca. 1000 cfu/mL nasal fluid. It's notable, however that the ALCARE® maintains a detectable presence in the nasal passage up to 5 days following injection. This may mean that the high counts penetrated to the lower respiratory areas and are still being cleared out 5 days later, or alternatively that the probiotics spore is able to establish a niche in the upper respiratory tract and compete with the normal flora present for “permanent residence”. Whether the relative amounts after the first day might have competitive exclusion properties vs. respiratory pathogens remains unknown. The notable features of this experiment may be that a) the cattle were reported to have taken the nasal treatments with no adverse effect, b) high probiotics counts can be established initially in the bovine nasal passage, and c) injected probiotics can be detected up to 5 days following injection, albeit at lower levels. Another observation to note from FIG. 1 is that the carrier-treated cattle appear to have consistently (but not statistically significant) lower total counts versus untreated or ALCARE® treated cattle. This could mean that the carrier had an affect of “flushing out” the nasal passage of endogenous flora. The higher ALCARE® counts at days 1, 3 and 5 would thus be representing the added probiotic taking up a greater percentage of the nasal passage, above that of carrier-only treated cattle. It can be speculated that the carrier itself could have some microbe-flushing/cleansing action that could potentially impact the balance of microbiota in the respiratory tract.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out the present invention, but that the invention will include all embodiments falling within the scope of the appended claims. Any combination of the above-described elements in all possible variations thereof is encompassed by the invention unless otherwise indicated herein or otherwise clearly contradicted by context.

All cited patents, patent applications, and other references are incorporated herein by reference in their entirety.

What is claimed is:

1. A method for the prevention and treatment of a respiratory infection in an animal comprising administering into the nasal passages of the animal a composition comprising an effective amount of one or more species of probiotic bacteria, and a suitable carrier for nasal administration.

2. The method according to claim 1, wherein the one or more species of probiotic bacteria are selected from the group consisting of non-pathogenic members of the Aspergillus, Bacillus, Bacteroides, Bifidobacterium, Lactobacillus, Leuconostoc, Pediococcus, Propionibacterium, Saccharomyces, and Enterococcus genus.

3. The method according to claim 1, wherein the one or more species of probiotic bacteria are acid-producing species of probiotic bacteria.

4. The method according to claim 3, wherein the one or more acid-producing species of probiotic bacteria are in the form of spores.

5. The method according to claim 4, wherein the one or more acid-producing species of probiotic bacteria are selected from the group consisting of Bacillus coagulans, and Bacillus licheniformis.

6. The method according to claim 4, wherein the one or more acid-producing species of probiotic bacteria are selected from the group consisting of Bacillus coagulans (ATCC No. 31284), Bacillus licheniformis (ATCC No. 10716), and Bacillus licheniformis (NCTC No. 13123).

7. The method according to claim 6, wherein the suitable carrier comprises sodium carboxymethylcellulose (CMC), and the composition is in the form of a suspension.

8. The method according to claim 1, wherein the composition further comprises one or more prebiotics.

9. The method according to claim 8, wherein the one or more prebiotics are selected from the group consisting of inulin, and fructo-oligosaccharides (FOS).

10. The method according to claim 1, wherein the suitable carrier is selected from the group consisting of glycerin, sodium CMC, methylcellulose, natural hydrocolloids, alginic acid, carageenan, locust bean gum, guar gum, gelatin, and clays.

11. The method according to claim 10, wherein the suitable carrier comprises sodium CMC, and the composition is in the form of a suspension.

12. A method for the prevention and treatment of a respiratory infection in an animal comprising administering into the nasal passages of the animal a composition comprising an effective amount of Bacillus licheniformis (NCTC No. 13123) bacteria, and a suitable carrier for nasal administration.

13. The method according to claim 12, wherein the suitable carrier comprises sodium CMC, and the composition is in the form of a suspension.

14. The method according to claim 13, wherein the carrier comprises from 0.1 to 1 w/v-% of sodium CMC.

15. The method according to claim 13, wherein the carrier comprises 0.25 w/v-% of sodium CMC.

16. The method according to claim 13, wherein the Bacillus licheniformis bacteria are in the form of spores.

17. The method according to claim 16, wherein the composition contains from 0.5×10⁸ to 1.5×10⁸ colony forming units (cfu) of spores per mL of the composition.

18. The method according to claim 16, wherein the method comprises administering from 1.0×10⁸ to 1.8×10⁹ cfu of spores per day.

19. The method according to claim 12, wherein the composition further comprises one or more prebiotics.

20. The method according to claim 19, wherein the one or more prebiotics are selected from the group consisting of inulin and fructo-oligosaccharides (FOS).
